(19) United States
(12) Patent Application Publication
(10) Pub. No.: US 2023/0113545 A1
(43) Pub. Date: Apr. 13, 2023

(54) ALERTS IN AMBULATORY INFUSION PUMP SYSTEMS

(71) Applicant: Tandem Diabetes Care, Inc., San Diego, CA (US)

(72) Inventor: Virginia S. Lu, San Diego, CA (US)

(21) Appl. No.: 17/961,206

(22) Filed: Oct. 6, 2022

Related U.S. Application Data
(60) Provisional application No. 63/253,397, filed on Oct. 7, 2021.

(51) Int. Cl. A61M 5/142

(52) U.S. Cl. CPC A61M 5/142; A61M 2205/502; A61M 2230/201; A61M 2205/18; A61M 2205/3569

(57) ABSTRACT

Disclosed herein are systems and methods for optimizing alarms, alerts, reminders and other notifications in an ambulatory infusion pump system. In an infusion pump system that includes a smartphone or other remote control device that interfaces with the infusion pump, such notifications can be given on both the remote control device and the infusion pump. To provide increased convenience and discretion for the user, the user may be able to opt to receive some or all notifications initially at only a single device, such as the smartphone, with some notifications, for example, being escalated to also be given at the pump if not acknowledged on the smartphone.

### RELATED APPLICATION

The present application claims the benefit of U.S. Provisional Application No. 63/253,397, filed Oct. 7, 2021, which is hereby incorporated herein in its entirety by reference.

### TECHNICAL FIELD

The present disclosure relates generally to ambulatory infusion pumps and, more particularly, to alerts, alarms and reminders issued by ambulatory infusion pump systems.

### BACKGROUND

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with type 1, or in some cases, type 2 diabetes. Some insulin injecting pumps are configured as portable or ambulatory infusion devices that can provide continuous subcutaneous insulin injection and/or infusion therapy as an alternative to multiple daily insulin injections via syringe or injector pen. Such ambulatory infusion pumps may be worn by the user, may use replaceable medicament cartridges, and may deliver other medicaments alone, or in combination with insulin. Such medicaments include glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

Ambulatory infusion pump systems can monitor a number of conditions relating to the pump and treatment with the pump. Such systems can therefore be configured to automatically provide various alerts, alarms and reminders to a user when a monitored condition requires user action or the user should otherwise be informed of the condition.

### SUMMARY

Disclosed herein are systems and methods for optimizing alarms, alerts, reminders and other notifications in an ambulatory infusion pump system. In an infusion pump system that includes a smartphone or other remote control device that interfaces with the infusion pump, such notifications can be given on both the remote control device and the infusion pump. To provide increased convenience and discretion for the user, the user may be able to opt to receive some or all notifications initially at only a single device, such as the smartphone, with some notifications, for example, being escalated to also be given at the pump if not acknowledged on the smartphone.

In an embodiment, an ambulatory infusion pump system can include a user-wearable infusion pump configured to deliver insulin to a user and a remote control device configured to remotely control the user-wearable infusion pump. At least one of the user-wearable infusion pump and remote control device can be configured to present a menu option to the user on a user interface to have at least some notifications first delivered by only one of the user-wearable infusion pump or the remote control device. If a user entry selecting one of the user-wearable infusion pump or the remote control device for first delivery of the at least some notifications is received, certain notifications can initially be provided on only the selected one of the user-wearable infusion pump or the remote control device. If, after a predetermined period of time, the user has not acknowledged a notification on the selected one of the user-wearable infusion pump or the remote control device, the notification can be provided on the other of the user-wearable infusion pump or the remote control device, or on both devices simultaneously.

### BRIEF DESCRIPTION OF THE DRAWINGS

The disclosure may be more completely understood in consideration of the following detailed description of various embodiments in connection with the accompanying drawings, in which:

FIG. 1 is an embodiment of an ambulatory infusion pump for use with embodiments of the disclosure.

FIG. 2 is a block diagram of the ambulatory infusion pump of FIG. 1.

FIGS. 3A-3B are an alternate embodiment of an ambulatory infusion pump for use with embodiments of the disclosure.

FIG. 4 depicts an embodiment of an infusion pump system according to the disclosure.

FIGS. 5A-5B depict remote control devices for an infusion pump system according to the disclosure.

FIG. 6 depicts a flowchart of methods steps for operation of an ambulatory infusion pump system according to an embodiment.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

The following detailed description should be read with reference to the drawings in which similar elements in different drawings are numbered the same. The drawings, which are not necessarily to scale, depict illustrative embodiments and are not intended to limit the scope of the disclosure.

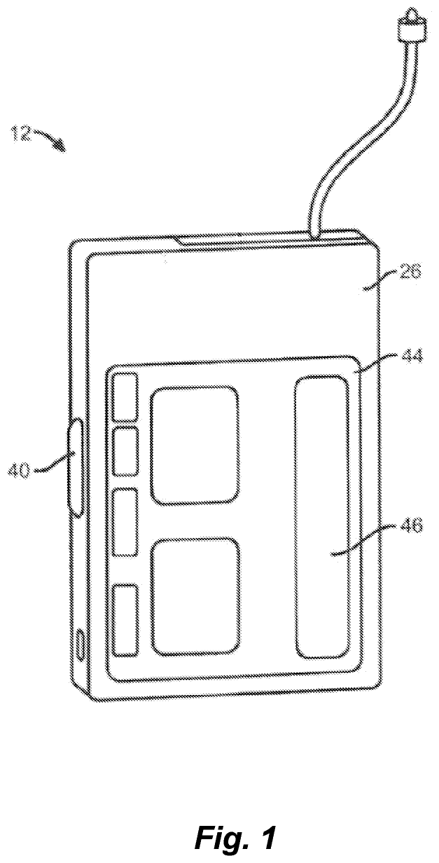

FIG. 1 depicts an example infusion pump that can be used in conjunction with one or more embodiments of the ambulatory infusion pump system of the present disclosure. Pump 12 includes a pumping or delivery mechanism and reservoir for delivering insulin or other medicaments to a patient and an output/display 44. The output/display 44 may include an interactive and/or touch sensitive screen 46 having an input device such as, for example, a touch screen comprising a capacitive screen or a resistive screen. The pump 12 may additionally or instead include one or more of a keyboard, a microphone or other input devices known in the art for data entry, some or all of which may be separate from the display. The pump 12 may also include a capability to operatively couple to one or more other display devices such as a remote display (e.g., a dedicated remote display or a CGM display), a remote control device, or a consumer electronic device (e.g., laptop computer, personal computer, tablet computer, smartphone, electronic watch, electronic health or fitness monitor, or personal digital assistant). Further details regarding such pump devices can be found in U.S. Pat. No. 8,287,495, previously incorporated by reference above. It is to be appreciated that pump 12 may be optionally configured to deliver one or more additional or other medicaments to a patient.

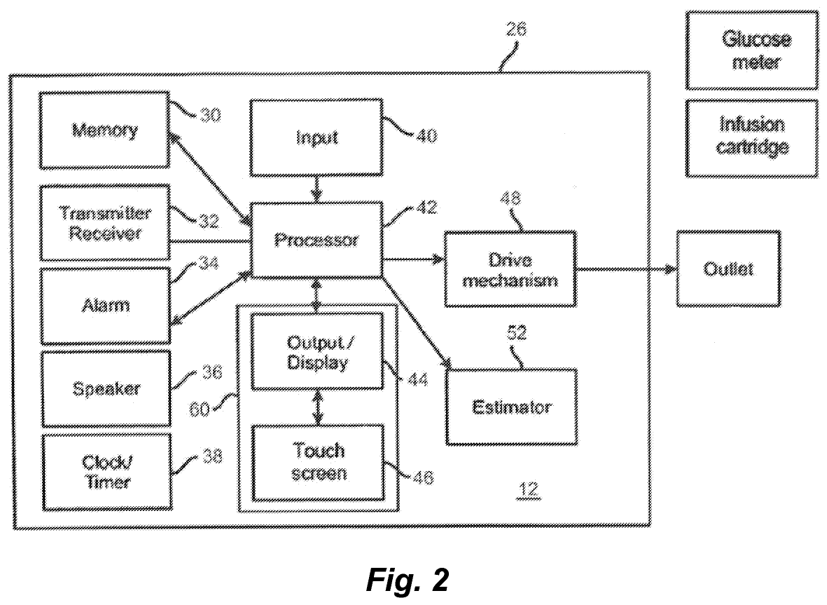

FIG. 2 illustrates a block diagram of some of the features that may be included within the housing 26 of pump 12. The pump 12 can include a processor 42 that controls the overall functions of the pump. The pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52 and a microphone (not pictured). One embodiment of a user interface is a graphical user interface (GUI) 60 having a touch sensitive screen 46 with input capability. In some embodiments, the processor 42 may communicate with one or more other processors within the pump 12 and/or one or more processors of other devices through the transmitter/receiver 32 such as a remote device (e.g., CGM device), a remote control device, or a consumer electronic device. In some embodiments, the communication is effectuated wirelessly, by way of example only, via a near field communication (NFC) radio frequency (RF) transmitter or a transmitter operating according to a “Wi-Fi” or Bluetooth® protocol, Bluetooth® low energy protocol or the like. The processor 42 may also be programmed to receive signals and/or other data from an input device, such as, by way of example, a pressure sensor, a temperature sensor, or the like.

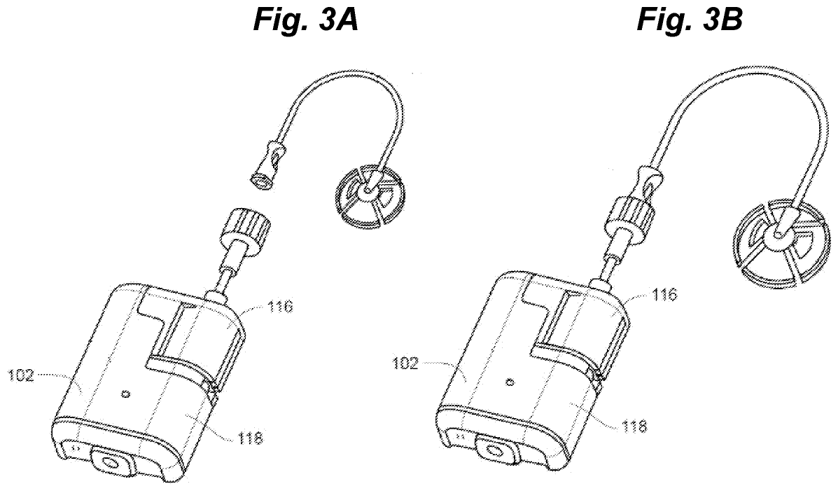

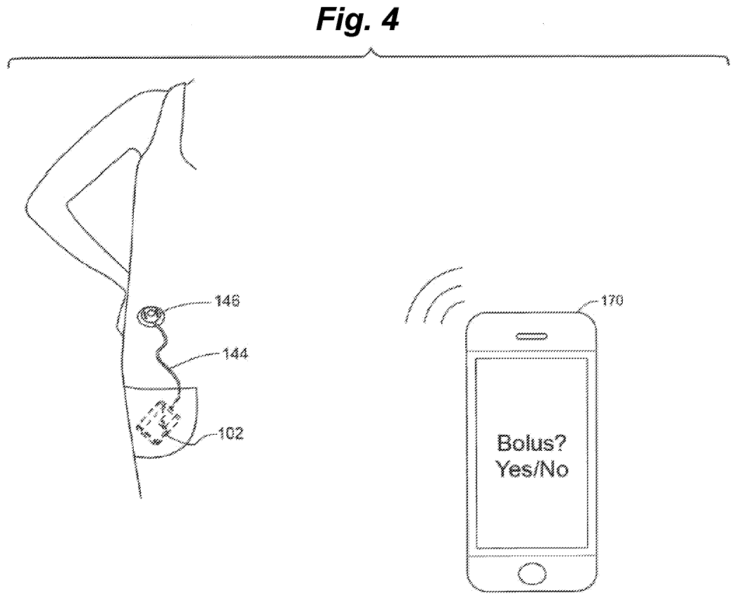

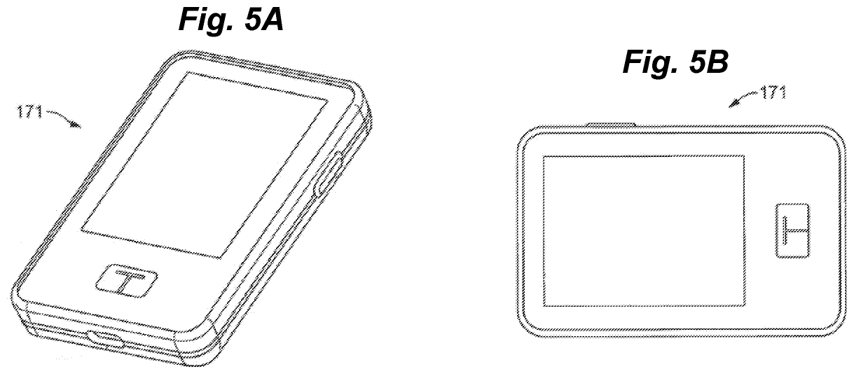

FIGS. 3A-3B depicts a second infusion pump that can be used in conjunction with one or more embodiments of the ambulatory infusion pump system of the present disclosure. Pump 102 includes a pump drive unit 118 and a medicament cartridge 116. Pump 102 also includes a processor that controls some or all of the operations of the pump. The processor may communicate with one or more processors within the pump 102 and/or one or more processors of other devices. The processor may also include programming to receive signals and/or other data from an input device, such as, by way of example, a pressure sensor, a temperature sensor, or the like. In some embodiments, pump 102 receive commands from a separate device for control of some or all of the operations of the pump. Such separate device can include, for example, a dedicated remote control device or a consumer electronic device such as a smartphone having a processor executing an application configured to enable the device to transmit operating commands to the processor of pump 102. In some embodiments, processor can also transmit information to one or more separate devices, such as information pertaining to device parameters, alarms, reminders, pump status, etc. Such separate device can include any remote display, remote control device, or a consumer electronic device as described above. Pump 102 can also incorporate any or all of the features described with respect to pump 12 in FIG. 2. In some embodiments, the communication is effectuated wirelessly. Further details regarding such pumps can be found in U.S. Pat. No. 10,279,106 and U.S. Patent Publication Nos. 2016/0339172 and 2017/0049957, each of which is hereby incorporated herein by reference in its entirety. Referring to FIGS. 4-5B, one or more remote control devices 170, 171 can be used to communicate with the processor of pump 12 or pump 102 to control delivery of medicament and transfer data with pump via a wired or a wireless electromagnetic signal, such as via, e.g., a near field communication (NFC) radio frequency (RF) modality or other RF modalities such as Bluetooth®, Bluetooth® low energy, mobile or Wi-Fi communication protocols, for example, according to embodiments of the present disclosure. Such a remote control can include, for example, a mobile communication device 170, such as a smartphone (as depicted in FIG. 4) executing a software application for control of the pump, a dedicated remote controller 171 (as depicted in FIGS. 5A-5B), a wearable electronic watch or electronic health or fitness monitor or personal digital assistant (PDA), etc., or a tablet, laptop or personal computer. Such communications between (and among) the one or more remote control devices 170, 171 and pump may be one-way or two-way for, e.g., effective transfer of data among the devices and the pump, control of pump operations, updating software on the devices and/or pump, and allowing pump-related data to be viewed on the devices and/or pump.

Embodiments of the present disclosure include components capable of and methods using wired and wireless transmission and receipt of signals for exchange of information and commands between and among any of the components as described herein, including, e.g., between a pump and a smartphone; among a pump, a CGM and a smartphone; between a dedicated remote controller and a pump; among a dedicated remote controller, a CGM and a pump; among a dedicated remote controller, a BGM and a pump, and other combinations as would be contemplated by those of skill in the art.

Ambulatory infusion pump systems as described herein can monitor a number of conditions relating to the pump and treatment with the pump including, for example, an amount of medicament in the pump, a battery level of the pump, infusion set usage, therapy delivered with the pump, glucose levels of the user obtained from a continuous glucose monitor (CGM), CGM sensor and transmitter expiration, and others. Such systems can therefore be configured to automatically provide various alerts, alarms and reminders to a user when a monitored condition requires user action, or the user should otherwise be informed of the condition. Such notifications can be given, for example, on a user interface of the pump, a remote control device, a smartphone configured to control the pump, a continuous glucose monitor or any other device in an ambulatory infusion pump system.

In embodiments in which a smartphone or other remote control device can be used to communicate with and/or control a pump, any alerts or reminders can be given by both the remote control and the pump. However, it would be advantageous to enable users to receive notifications on a single device and escalate notifications to an additional device as needed.

In an embodiment, a flexible annunciation feature can provide increased convenience and/or discretion for a user by enabling at least some alerts, alarms, reminders, etc. to first be delivered to a user's device of choice. For example, a user may be able to opt-in to first receive notifications on the user's smartphone. If the user is unable to respond on the smartphone within a certain timeframe, some notifications can then be escalated to the user's infusion pump. In some embodiments, all notifications can first go to the chosen device, i.e., smartphone or remote control. In other embodiments, certain notifications that are considered critical can still be given on both the smartphone and the pump. For example, critical alerts such as a critical low glucose alert that the user's current and/or predicted glucose level is below a critically low threshold can require dual notification whereas non-critical notifications can be delivered first to the user's chosen device. In some embodiments, some notifications can be provided only on the chosen device and not escalated to the pump whether or not the notification is acknowledged on the first device.

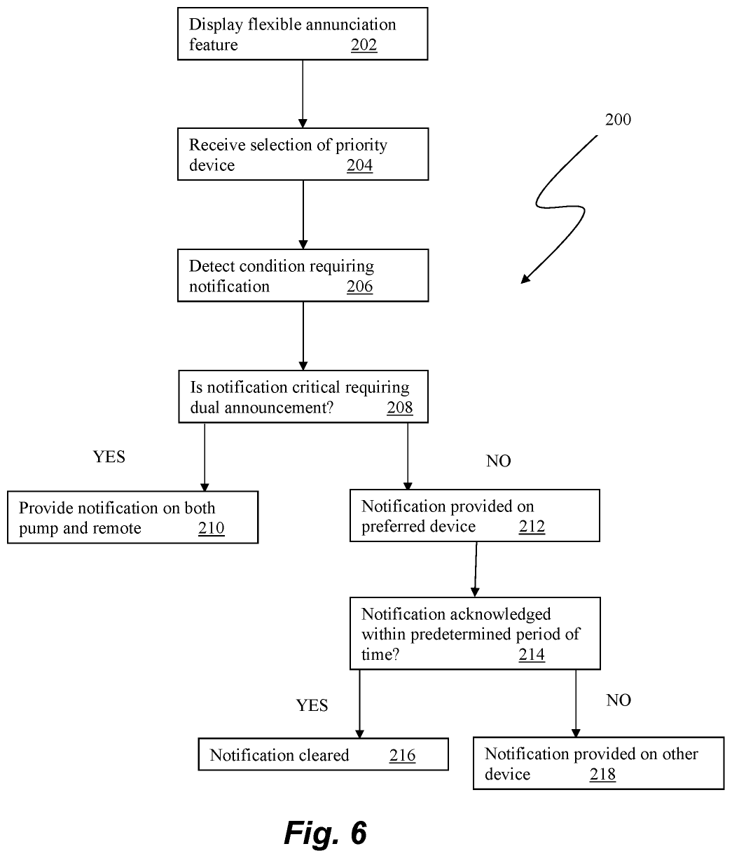

Referring now to FIG. 6, a flowchart of method steps 200 for operation of an ambulatory infusion pump system according to an embodiment is depicted. At step 202 a flexible annunciation feature is accessed by the user on a pump and/or remote control and at step 204 the user selects a preferred device to be given priority to first receive notifications. At step 206, a condition requiring a notification for the user is detected. The system optionally determines at step 208 whether the notification is a critical notification that requires the notification to be given on both the pump and the remote control device. If the notification is a critical notification, it can be provided on both the pump and the remote control at step 210. If the notification is not critical, the flexible annunciation feature can be employed and the notification can be provided at step 212 on the user's preferred device. The system can then determine whether or not the notification is acknowledged on the preferred device within a predetermined period of time at step 214. If the notification is acknowledged within the time period, the notification can be cleared at step 216 and not issued on the other device. If the notification is not acknowledged within the predetermined period, the notification can then be given on the user's other device at step 218. In some embodiments, some notifications, such as information or particularly non-critical notifications, may be cleared and not given on the other device even if not acknowledged on the preferred device.

In some embodiments, the system can therefore define and store various tiers of notifications that can have different notifications procedures. For example, there may be a first tier of critical notifications that are automatically provided on both the remote control device and the pump, a second tier of notifications that can first be provided on a preferred device and then escalated to the other device of not acknowledged and a third tier of information notifications that are not escalated even if they are not acknowledged on the preferred device.

In some embodiments, notifications can be provided based on a schedule of the user. For example, during certain times of the day some or all notifications can be preprogrammed to be given with one annunciation protocol (e.g., with the pump and the remote) while at other times of day another annunciation protocol (e.g., remote only) can be used for the same notifications. Annunciation protocols can also be tied to calendar events of the user, which may be preprogrammed or otherwise obtained from a smartphone or other user device. For example, certain notifications that may be given on both the pump and remote control may be given on only the remote control when the user has a meeting scheduled or other event where the user may want to limit disruption. Similarly, certain notifications could be silenced during such events or delayed and provided after such events. Annunciation protocols can also be varied based on a mode of use of the pump. For example, annunciation protocols may be different during a normal activity, exercise activity mode (i.e., the user is exercising), sleep mode (i.e., the user is sleeping) or during other personalized profile settings. Annunciation protocols can also be varied based on whether the pump is being operated in closed loop mode or open loop mode.

In embodiments, an ambulatory infusion pump system can include a user-wearable infusion pump configured to deliver insulin to a user and a remote control device configured to remotely control the user-wearable infusion pump. At least one processor can be configured to present a menu option to the user on a user interface to have at least one notification first delivered by only one of the user-wearable infusion pump or the remote control device, receive a user entry selecting one of the user-wearable infusion pump or the remote control device for first delivery of the at least one notification and to provide a notification to the selected one of the user-wearable infusion pump or the remote control device. If, after a predetermined period of time, the user has not acknowledged the notification on the selected one of the user-wearable infusion pump or the remote control device, provide the notification on the other of the user-wearable infusion pump or the remote control device.

In some embodiments, the user interface is part of the remote control device.

In some embodiments, the user interface is part of the user-wearable infusion pump.

In some embodiments, the remote control device is a smartphone.

In some embodiments, the processor is further configured to provide some notifications on both the remote control device and the user-wearable infusion pump simultaneously.

In some embodiments, the notifications provided on both the remote control device and the user-wearable infusion pump simultaneously are critical notifications.

In some embodiments, the processor is configured to provide some notifications on only the selected one of the user-wearable infusion pump or the remote control device and to not provide those notifications on the other of the user-wearable infusion pump or the remote control device even if the user has not acknowledged the notification with the predetermined period of time.

In some embodiments, the processor is further configured to store a default selection for the one of the user-wearable infusion pump or the remote control device to first deliver the at least one notification.

In some embodiments, the menu option enables the user to select the other of the user-wearable infusion pump or the remote control device from the default selection.

In some embodiments, the default selection is the remote control device.

In embodiments, an ambulatory infusion pump system can include a user-wearable infusion pump configured to deliver insulin to a user and a remote control device configured to remotely control the user-wearable infusion pump. At least one processor configured to store a preference to have at least one notification first delivered by one of the user-wearable infusion pump or the remote control device for first delivery of the at least one notification and to provide the at least one notification to the one of the user-wearable infusion pump or the remote control device. If, after a predetermined period of time, the user has not acknowledged the at least one notification on the one of the user-wearable infusion pump or the remote control device, provide the at least one notification on the other of the user-wearable infusion pump or the remote control device.

In some embodiments, the preference is stored in a memory of the remote control device.

In some embodiments, the preference is stored in a memory of the user-wearable infusion pump.

In some embodiments, the remote control device is a smartphone.

In some embodiments, the processor is further configured to provide some notifications on both the remote control device and the user-wearable infusion pump simultaneously.

In some embodiments, the notifications provided on both the remote control device and the user-wearable infusion pump simultaneously are critical notifications.

In some embodiments, the processor is configured to provide some notifications on only the one of the user-wearable infusion pump or the remote control device and to not provide those notifications on the other of the user-wearable infusion pump or the remote control device even if the user has not acknowledged the notification with the predetermined period of time.

In some embodiments, the processor is further configured to store a default preference for the one of the user-wearable infusion pump or the remote control device to first deliver the at least one notification.

In some embodiments, the at least one processor is further configured to display a menu option that enables the user to select the other of the user-wearable infusion pump or the remote control device from the default preference.

In some embodiments, the default preference is the remote control device.

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; 11,305,057; 11,458,246; and 11,464,908 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0368430; 2020/0372995; 2021/0001044; 2021/0113766; 2021/0154405; 2021/0353857; 2022/0062553; 2022/0139522; 2022/0223250; 2022/0233772; 2022/0233773; 2022/0238201; and 2022/0265927 and commonly owned U.S. patent application Ser. Nos. 17/368,968; 17/729,464; 17/732,208; 17/878,681; 17/879,959; 17/886,998 and 17/896,492.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. An ambulatory infusion pump system, comprising:
a user-wearable infusion pump configured to deliver insulin to a user; and
a remote control device configured to remotely control the user-wearable infusion pump,
at least one processor configured to:
present an option to the user on a user interface to have at least one notification delivered by only one of the user-wearable infusion pump or the remote control device;
receive a user entry selecting one of the user-wearable infusion pump or the remote control device for delivery of the at least one notification;
provide a notification to the selected one of the user-wearable infusion pump or the remote control device;
if, after a predetermined period of time, the user has not acknowledged the notification on the selected one of the user-wearable infusion pump or the remote control device, provide the notification on the other of the user-wearable infusion pump or the remote control device.

2. The system of claim 1, wherein the user interface is part of the remote control device.

3. The system of claim 1, wherein the user interface is part of the user-wearable infusion pump.

4. The system of claim 1, wherein the remote control device is a smartphone.

5. The system of claim 1, wherein the at least one processor is further configured to provide notifications on both the remote control device and the user-wearable infusion pump simultaneously.

6. The system of claim 5, where the notifications provided on both the remote control device and the user-wearable infusion pump simultaneously are critical notifications.

7. The system of claim 1, wherein the at least one processor is configured to provide some notifications on only the selected one of the user-wearable infusion pump or the remote control device and to not provide those notifications on the other of the user-wearable infusion pump or the remote control device even if the user has not acknowledged the notification with the predetermined period of time.

8. The system of claim 1, wherein the at least one processor is further configured to store a default selection for the one of the user-wearable infusion pump or the remote control device to first deliver the at least one notification.

9. The system of claim 8, wherein the option enables the user to select the other of the user-wearable infusion pump or the remote control device from the default selection.

10. The system of claim 8, wherein the default selection is the remote control device.

11. An ambulatory infusion pump system, comprising:
a user-wearable infusion pump configured to deliver insulin to a user; and
a remote control device configured to remotely control the user-wearable infusion pump,
at least one processor configured to:
store a preference to have at least one notification delivered by one of the user-wearable infusion pump or the remote control device for first delivery of the at least one notification;
provide the at least one notification to the one of the user-wearable infusion pump or the remote control device;
if, after a predetermined period of time, the user has not acknowledged the at least one notification on the one of the user-wearable infusion pump or the remote control device, provide the at least one notification on the other of the user-wearable infusion pump or the remote control device.

12. The system of claim 11, wherein the preference is stored in a memory of the remote control device.

13. The system of claim 11, wherein the preference is stored in a memory of the user-wearable infusion pump.

14. The system of claim 11, wherein the remote control device is a smartphone.

15. The system of claim 11, wherein the at least one processor is further configured to provide notifications on both the remote control device and the user-wearable infusion pump simultaneously.

16. The system of claim 15, where the notifications provided on both the remote control device and the user-wearable infusion pump simultaneously are critical notifications.

17. The system of claim 11, wherein the at least one processor is configured to provide some notifications on only the one of the user-wearable infusion pump or the remote control device and to not provide those notifications on the other of the user-wearable infusion pump or the remote control device even if the user has not acknowledged the notification with the predetermined period of time.

18. The system of claim 11, wherein the at least one processor is further configured to store a default preference for the one of the user-wearable infusion pump or the remote control device to first deliver the at least one notification.

19. The system of claim 18, wherein the at least one processor is further configured to display an option that enables the user to select the other of the user-wearable infusion pump or the remote control device from the default preference.

20. The system of claim 18, wherein the default preference is the remote control device.
